(19) United States
(12) Patent Application Publication
(10) Pub. No.: US 2002/0115961 A1
(43) Pub. Date: Aug. 22, 2002

(54) Hydration system for the disabled

(76) Inventor: Michael E. Writt SR., Indianapolis, IN (US)

Correspondence Address: Charles P. Schmal, Woodard, Emhardt, Naughton, Moriarty and McNett, Bank One Center/Tower, 111 Monument Circle, Suite 3700, Indianapolis, IN, 46204-5137, US

(21) Appl. No.: 10/121,173

(22) Filed: Apr. 10, 2002

Related U.S. Application Data
(63) Continuation-in-part of application No. 29/135,682, filed on Jan. 17, 2001, now Pat. No. D458846.
(60) Provisional application No. 60/283,084, filed on Apr. 11, 2001.

(51) Int. Cl. A61M 1/00

(52) U.S. Cl. 604/28

(57) ABSTRACT

A hydration system includes a container, a hose and a dispenser. The hose fluidly couples the container to the dispenser. The dispenser is adapted to control fluid flow to a patient and has a strap to hold the dispenser to the hand of the patient. The system further includes a hanger assembly adapted for repositioning the container and the hose in order to minimize kinking in the hose.

### REFERENCE TO RELATED APPLICATIONS

This application claims the benefit of U.S. Provisional Patent Application Serial No. 60/283,084, filed Apr. 11, 2001, and is a continuation-in-part of U.S. Design Patent Application Serial No. 29/135,682, filed Jan. 17, 2001, both of which are hereby incorporated by reference in their entirety.

### FIELD OF THE INVENTION

The present invention generally relates to hydration systems, and more specifically, but not exclusively, concerns a hydration system constructed and arranged to allow patients with limited mobility to hydrate themselves.

### BACKGROUND OF THE INVENTION

Hydration of a patient is a constant concern for health care providers. If a patient does not receive the requisite amount of fluids, the patient can become dehydrated which can lead to additional complications that can hamper recovery. One solution to this problem has been to supply fluids to the patient intravenously. However, due to infection risks and other concerns, intravenous hydration may not always be appropriate. An alternative solution has been to instruct the patient to drink water. Typically, a patient is supplied with a cup of water, or some other hydrating fluid, and instructed to drink. However, after an operation or due to disability, drinking from a cup can be rather difficult for the patient, and spills can frequently occur. In addition, the hydrating fluid may not be available for the patient to drink, if the nurse or other healthcare provider is unavailable to supply refills.

In an alternative approach, a tube or a straw is attached to a fluid container so as to reduce accidental spills and increase the fluid supply. A hand-operated valve is placed on a fluid dispensing end of the tube in order to prevent leakage. To drink the fluid, the patient must actuate the valve by hand. Operation of such a valve can be difficult when the patient is extremely weak or has some hand dexterity problems due to a medical condition, such as partial paralysis. For such patients, the task of grasping of the valve and holding the valve to their mouth can be difficult. The more difficulty the patient experiences in trying to drink, the less likely the patient will properly hydrate themselves. To remedy this situation, an alternative approach uses a valve on which the patient bites down in order to drink the fluid. In this approach, the valve is placed near the mouth of the patient so that the patient needs only to move their head to reach the valve. The patient bites down on the valve in order to drink the fluid. However, having the valve constantly near their face may annoy the patient. Such valve designs can be prone to leakage when not in use. Furthermore, these designs have not addressed the problem of fluid leaking from the patient's mouth during drinking. Patients, due to their weakened condition, may not be always be able contour their lips in order to match the shape of the valve so as to minimize fluid leakage from their mouth. This fluid leakage from the patient's mouth during drinking can make the patient wet and uncomfortable, and this in turn, may reduce the patient's desire to drink.

Another concern with fluid delivery systems is the risk that the fluid supply hose will become kinked and prevent the patient from drinking fluid. If the hose is improperly located, the patient or some other person may accidentally pinch or kink the hose so as to cut off the fluid supply. This problem can be exacerbated when the patient is transported to a different location. Hooks for hanging the fluid container may not always be available. Consequently, the health care provider resorts to laying the container on a table or some other object, and if not careful, the supply hose can become pinched underneath the container so as to cut-off the fluid supply.

Therefore, there remains a need for a hydration system that can be easily used by patients with limited hand mobility and which can dependably deliver fluid with minimal fluid leakage.

### SUMMARY OF THE INVENTION

One embodiment of the present invention is directed to a unique hydration system. Other embodiments include, for example, unique apparatus and methods for delivering fluid to a person.

A further embodiment pertains to a hydration system. The hydration system includes a hose adapted to supply fluid. A fluid delivery device is coupled to the hose and is adapted to control fluid flow. The delivery device has a hand strap to secure the device to the hand of a patient.

Another embodiment concerns a hydration system. The hydration system includes a fluid container adapted to store a fluid and a hose fluidly coupled to the container. A fluid dispenser is fluidly coupled to the hose, and the dispenser includes a valve. The valve has a valve seat and a valve flap. The flap has one or more openings defined therein. The valve seat is adapted to close the openings when the valve is closed, and the valve flap is adapted to deflect away from the valve seat when the valve is opened to allow the fluid to flow through the openings.

A further embodiment concerns a hydration system that includes a fluid container and a fluid supply hose coupled to the fluid container. A fluid dispenser is coupled to the hose, and the dispenser is constructed and arranged to control fluid flow from the container to a person with a hand. A hand strap is coupled to the fluid dispenser in order to secure the fluid dispenser to the hand of the person.

A further embodiment concerns a fluid dispenser. The fluid dispenser includes a mouthpiece that defines a fluid passage. The mouthpiece includes a deformable wall that extends across the fluid passage. The deformable wall defines at least one fluid opening in the mouthpiece that is adapted to dispense fluid to a person. A valve seal is provided within the fluid passage to seal the fluid opening when the deformable wall is undeflected from the valve seal and to allow the fluid to flow through the fluid opening when the deformable wall is deflected away from the valve seal.

Another embodiment concerns a hanger assembly that includes a base. The base includes a connector wall and a pair of opposing side walls that extend from the connector wall to straddle a support. The base further includes a clamping mechanism adapted to secure the base to the support and to allow lateral positional adjustment along the support. The base defines a bore and a plurality of radially extending slots. A riser includes a rod and a pair of opposing pins extending from the rod. A portion of the rod with the pins is received in the bore, and the pins are received in the slots to provide incremental rotational adjustment of the riser. A hook is coupled to the riser to support a fluid container.

A further embodiment concerns a fluid container for hydrating a patient that includes a hollow container body adapted to contain a fluid. The body includes a pair of supports with each having flat support surfaces to stably support the body on a support surface. The pair of supports define an outlet cavity, and a nozzle extends in a radial direction in the outlet cavity between the pair of supports. The nozzle is adapted to couple to a hose. A hanger is coupled to the body to hang the body.

One object of the present invention as provided improved hydration delivery system. Related objects and advantages of the present invention will be apparent from the following description, claims, and drawings.

### BRIEF DESCRIPTION OF THE DRAWINGS

FIG. 1 is a perspective view of a hydration system according to one embodiment of the present invention.

FIG. 2 is a side view of a fluid dispenser used in the FIG. 1 hydration system.

FIG. 3 is an end view of one end of the FIG. 2 fluid dispenser.

FIG. 4 is an end view of the other end of the FIG. 2 fluid dispenser.

FIG. 5 illustrates a patient using the FIG. 1 hydration system.

FIG. 6 is a cross-sectional view of the FIG. 2 fluid dispenser in full section.

FIG. 7 illustrates a cross-sectional view of a mouthpiece valve of the FIG. 2 fluid dispenser in a closed position.

FIG. 8 illustrates a cross-sectional view of the mouthpiece valve in an opened position.

FIG. 9 illustrates an exploded view of a support assembly used in the FIG. 1 hydration system.

FIG. 10 illustrates a top view of a clamping mechanism used in the FIG. 9 support assembly.

FIG. 11 illustrates a side view of the FIG. 10 clamping mechanism.

FIG. 12 is a perspective view of a fluid container according to another embodiment of the present invention.

FIG. 13 is an perspective view of one end of the FIG. 12 container.

FIG. 14 illustrates a perspective view of a hydration system with the FIG. 12 fluid container.

FIG. 15 is a side view of a top portion of the FIG. 12 container.

FIG. 16 is a perspective view of a portion of a hanger used with the FIG. 12 container.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

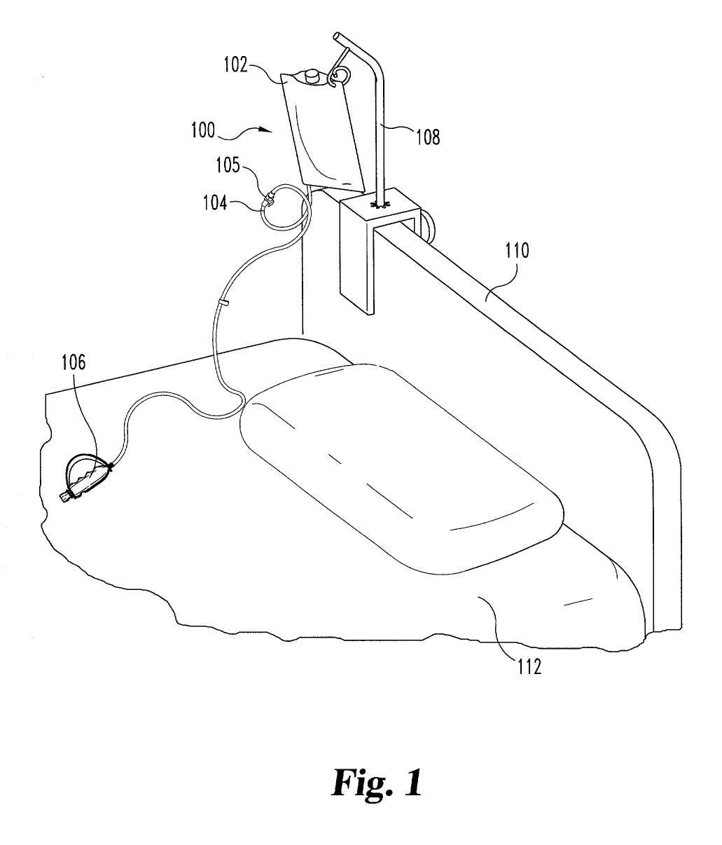

While the invention will be described below for with reference to a medical setting, it should be appreciated that the present invention can be adapted for use in other situations where hydration is a concern. FIG. 1 illustrates a hydration system 100 according to one embodiment of the present invention. Hydration system 100 includes a fluid container 102, a flexible hose (tubing) 104 with a quick connect/disconnect connection 105 and a fluid dispenser 106. As shown, fluid delivery system 100 is supported with a support assembly 108. Support assembly 108 positions fluid container 102 above a patient so that hose 104 is properly pressurized for dispensing fluid to the patient. In the illustrated embodiment, support assembly 108 is attached to a headboard 110 of a bed 112. It should be appreciated that support assembly 108 can be attached to other types of structures. Support assembly 108 is adapted to be movable along head board 110 so as to adjust the position of fluid delivery system 100. Hose 104 is fluidly coupled to container 102 and fluid dispenser 106. Hose 104 includes connection 105 that is adapted to easily disconnect dispenser 106 from container 102. This allows each patient to have their own fluid dispenser 106, which can improve hygiene. Connection 105 includes a valve that prevents fluid from leaking when dispenser 106 is disconnected from container 102. In one form, connection 105 is a CPC valve manufactured by Colder Manufacturing. Fluid container 102 is adapted to store hydrating fluids, such as water. In one form, fluid container 102 is a clear or semitransparent bag with fluid level indicators. In another form, fluid container 102 is an opaque (blue) bag with a clear view strip portion in which the fluid level can be viewed. Hose 104 is attached to container 102 in order to deliver the fluid from container 102 to fluid dispenser 106.

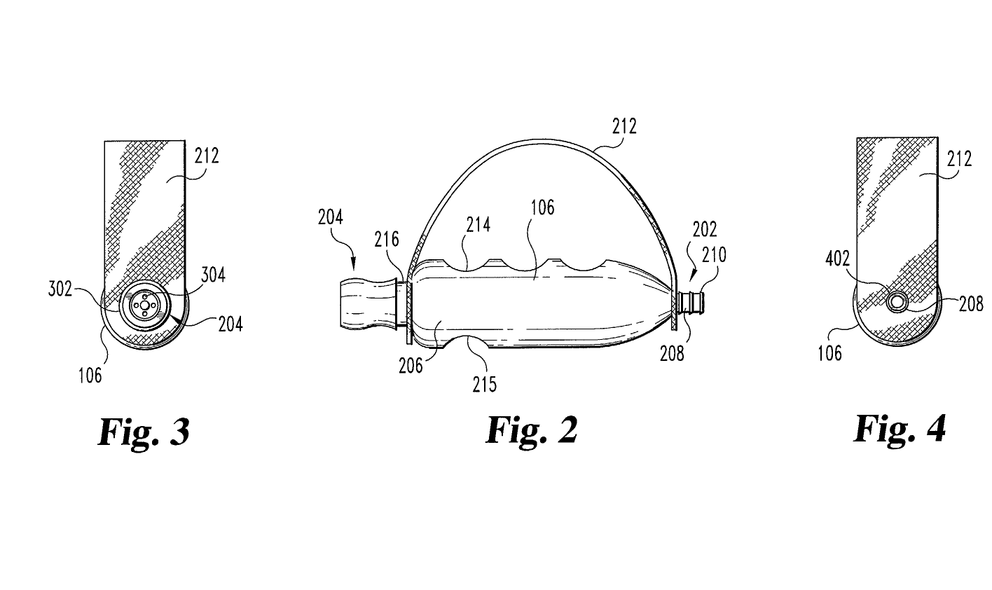

Fluid dispenser 106 is illustrated in further detail in FIGS. 2-4. As shown in FIG. 2, fluid dispenser 106 has a connection end portion 202 and an oppositely provided fluid dispensing end portion 204. A handle portion 206 is provided between end portions 202 and 204. Connection end portion 202 has a connector 208 which is used to quickly connect and disconnect tubing 104 from fluid dispenser 106. Connector 208 has one or more sealing ridges that secure and seal hose 104. As shown, hand strap 212 spans handle portion 206. Handle portion 206 includes a plurality of finger indentations 214 adapted to engage the fingers of a patient (person) and an opposite thumb indentation 215 adapted to coincide with the contour of the thumb of the patient. In one form, handle portion 206 is formed of plastic in order to provide a comfortable and yet secure grip for the patient. Between fluid dispensing end portion 204 and handle portion 206, fluid dispenser 106 has a handle strap adjustment portion 216 around which strap 212 slides. Strap adjustment portion 216 has a substantially smooth cylindrical shape that allows hand strap 212 to move laterally in order to adjust for differently sized hands. In one embodiment, hand strap 212 is made with an adjustable VELCRO brand type strip in order to allow adjustment for varying hand sizes. Hand strap 212 allows a patient with limited hand mobility to grip and hold onto fluid dispenser 106. If the hand of the patient is weak and/or becomes fatigued, hand strap 212 prevents fluid dispenser 106 from falling off the hand of the patient.

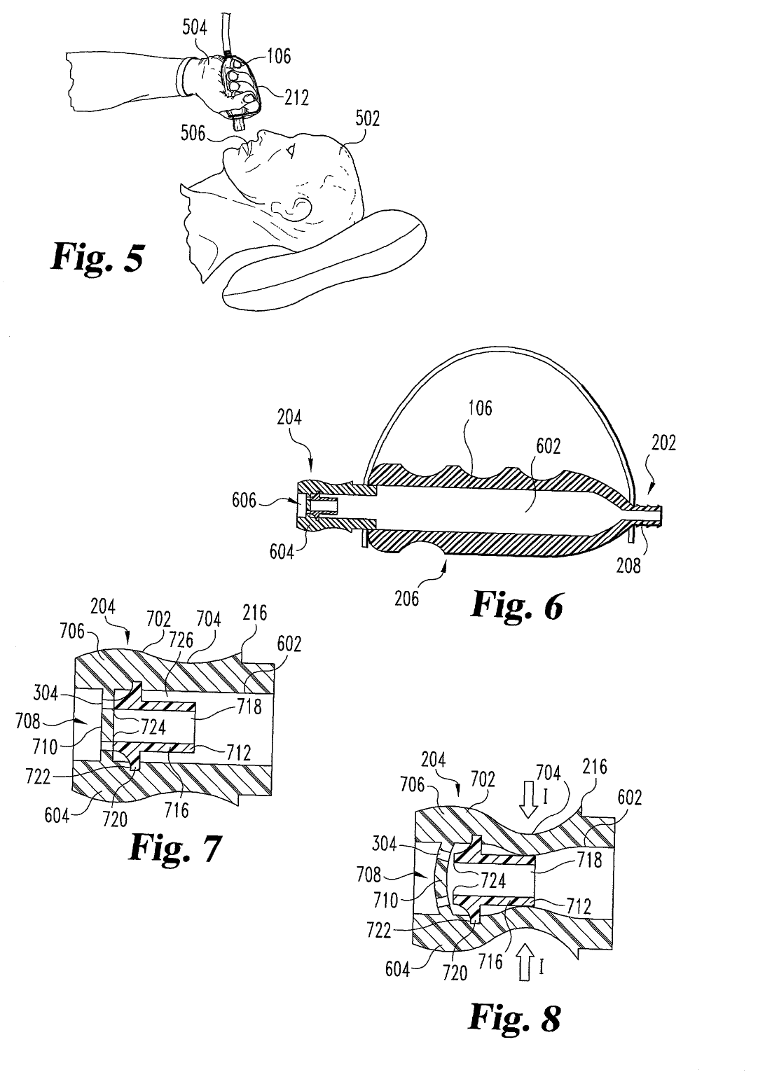

As shown in FIGS. 3 and 4, hand strap 212 includes a pair of holes 302 and 402 which are provided to allow the position of hand strap 212 to be rotatably adjustable for the patient. This allows fluid dispenser 106 to be securely held by the patient at multiple orientations. For instance, hand strap 212 is rotatably adjustable to rotate about handle portion 206 to allow the patient to hold dispenser 106 in either their left or right hand. The patient can also hold dispenser 106 with fluid dispensing end portion 204 directed towards or away from the patient's thumb. Moreover, hand strap 212 can be secured over either the patient's thumb or middle fingers. As illustrated in FIG. 3, fluid dispensing portion 204 includes one or more openings 304 that are adapted to dispense fluid. In the illustrated embodiment, fluid dispensing portion 204 has four (4) radially disposed openings 304 that are equidistantly spaced. In use, as shown in FIG. 5, hand strap 212 along with indentations 214 and 216 allow a patient 502 to easily hold fluid dispenser 106 in their hand 504 and raise fluid dispenser 106 to their mouth 506. Once patient 502 has completed drinking, patient 502 can move dispenser 106 away from their mouth 506.

As illustrated in FIG. 6, fluid dispenser 106 has a fluid passage 602 defined therein that allows fluid to pass from connection portion 202 to fluid dispensing portion 204. Fluid dispensing portion 204 includes a mouthpiece 604 at which patient 502 receives fluid from mouthpiece opening 606. Mouthpiece 604, as shown in FIGS. 7-8, has a contoured outer surface 702 that includes a bite portion 704 and bulbously shaped seal portion 706. Patient 502 can bite on bite portion 704 and/or suck on mouthpiece 604 to receive fluid from dispenser 106. Seal portion 706 has a larger outer diameter than bite portion 704 such that during drinking the bulbous shape of portion 706 matches the contour of mouth 506 of patient 502 so as to prevent fluid leakage from the mouth during drinking. Mouthpiece 604 includes a valve 708 that is used to control fluid flow and to seal fluid dispenser 106. Valve 708 includes a deformable wall or flap 710 and valve seal 712. As illustrated in FIG. 7, flap 710 spans across passage 602, and flap 710 includes one or more openings 304 through which fluid passes when valve 708 is opened (FIG. 8). In the illustrated embodiment, flap 710 has four openings 304 that are spaced around flap 710 (FIG.3). In order to provide comfort, mouthpiece 604 is made of flexible material. Likewise, flap 710 is made of flexible material so that flap 710 is able to deflect away from valve seal 712. In one form, mouthpiece 604 is made of a silicone material, which makes mouthpiece 604 more comfortable to patient 502.

Valve seal 712 has a valve seal wall 716 that defines a valve seal fluid passageway 718. In one form, valve seal 712 has a substantially cylindrical shape with passage 718 likewise being substantially cylindrical. Valve seal 712 further includes a flange 720 that is provided in slot 722 of mouthpiece 204 and a valve sealing end portion 724 which is positioned to seal holes 304 when valve 708 is closed (FIG. 7). In the illustrated cylindrical form, valve seal 712 is used to seal radially disposed fluid openings 304. Flanges 720 extend from valve seal 712 such that a gap 726 is formed between wall 716 and bite portion 704.

FIG. 7 illustrates valve 708 in a closed position, and FIG. 8 shows valve 708 in an opened position. When patient 502 places mouthpiece 204 in their mouth 506, patient 502 can withdraw fluid in two manners. In the first manner, patient 502 can suck on mouthpiece 204 to form low pressure in mouthpiece opening 606 which in turn causes flap 710 to deflect away from valve seal 712 such that fluid passes through passage 718 and out openings 304 into their mouth 506. In the second manner, as shown in FIG. 8, patient 502 bites on bite portion 704 in radially inward direction I. Flanges 720 provide a fulcrum for pivoting portions of mouthpiece 704 such that flap 710 deflects away from valve seal 712. Gap 726 allows bite portion 704 to deflect radially inward in direction I. When flap 710 is deflected, fluid passes through openings 304 and out mouthpiece opening 606. During the biting, walls 716 of valve seal 712 prevent bite portion 704 from collapsing passageway 602, and this allows fluid to flow through valve seal passageway 718. At the same time, bulbous portion 706 is sealed against the lips of patient 502 such that the fluid does not leak from their mouth 506. The above-described structure even allows the elderly without teeth to drink. Elderly patients without teeth can suck on mouthpiece 204 to drink and/or bite on bite portion 704 with their lips or gums in order to drink. Once patient 502 quits biting and/or sucking on mouthpiece 204, flap 710 returns to an undeflected position, as shown in FIG. 7, such that openings 304 are sealed against valve seal portion 724. Valve 708 is configured to allow adequate sealing in order to prevent seepage of fluid from fluid dispenser 106 when closed.

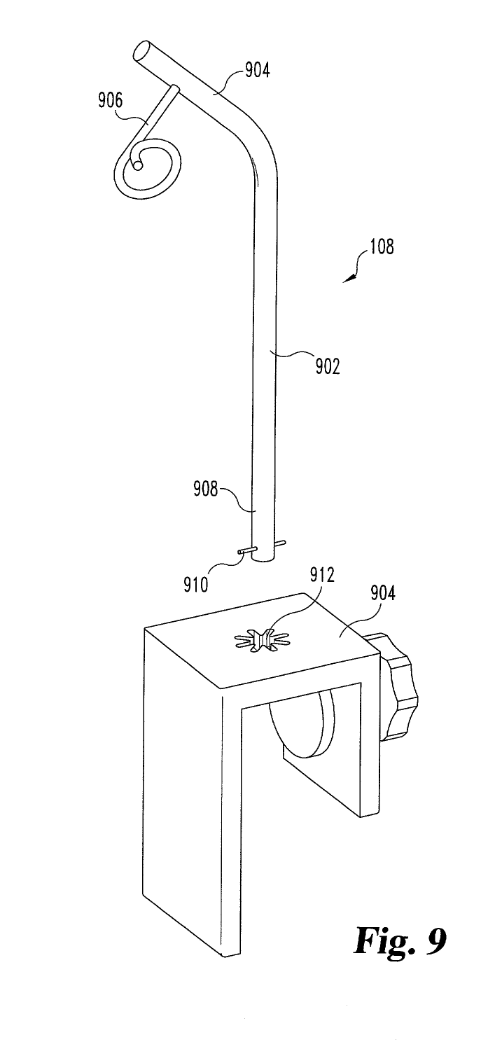

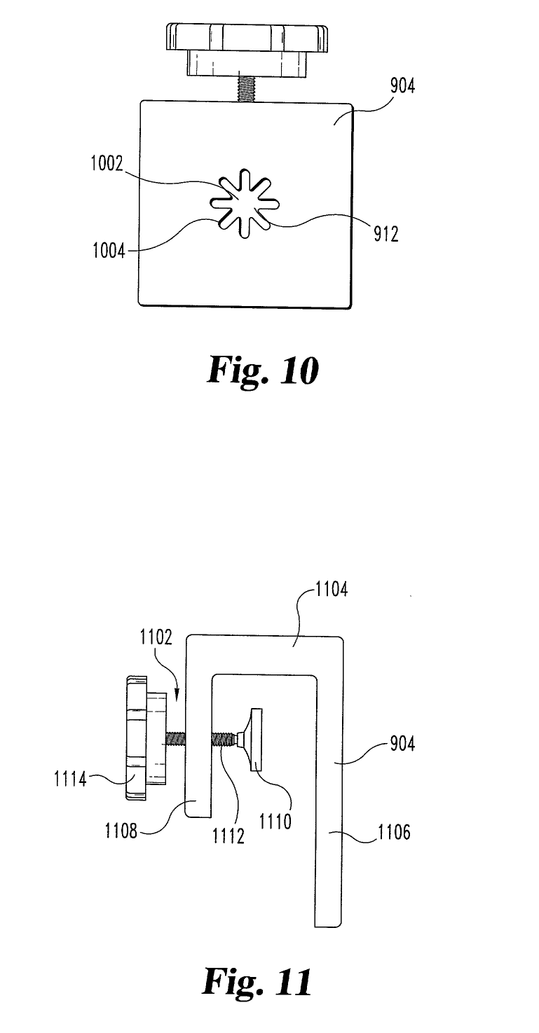

In the illustrated hydration system 100, a gravity feed arrangement is used in supplying the fluid. If hydration system 100 is improperly positioned, hose 104 may become kinked which can prevent the fluid from flowing. To minimize this risk, hanger assembly 108 allows for repositioning of hydration system 100 both laterally and rotationally. This ability to incrementally positioning of hydration system 100 reduces the likelihood kinking of hose 104 during drinking. As shown in exploded form in FIG. 9, hanger assembly 108 includes a riser 902 and a base 904. Riser 902 includes a bent extension portion 904 that has a hook 906 attached thereto. At the other end of riser 902, coupling portion 908 has a pair of positioning pins 910 extending therefrom. In the illustrated embodiment, riser 902 is a bent cylindrical rod. Coupling portion 908 along with positioning pins 910 are inserted into positioning opening 912 in order to couple riser 902 to base 904. As shown in further detail in FIG. 10, opening 912 includes a central opening portion 1002 with a plurality of radial slots 1004 radially extending therefrom. Central opening 1002 is sized to receive coupling portion 908, and slots 1004 are oriented to receive pins 910. Rotational orientation of riser 902 can be adjusted by repositioning pins 910 into different slots 1004. This allows assembly 108 to minimize obstructions to patient 502 and position tubing 104 so as to avoid kinking.

As shown in FIG. 11, base 904 has a U-shaped profile. Base 904 includes a clamping mechanism 1102 that secures base 904 with an object, such as headboard 110. Base 904 further includes an upper wall 1104 connected between two opposing side walls 1106 and 1108. As illustrated, clamping mechanism 1102 is threadedly coupled to wall 1108. Clamp 1102 includes a clamping portion 1110 coupled to a threaded portion 1112 which in turn is fixed to knob 1114. Knob 1114 is turned in one direction in order to clamp base 904 to a support, and knob 1114 is turned in the opposite direction in order to unclamp base 904. Clamping mechanism 1102 allows for quick and easy lateral adjustment of the position of assembly 108 along headboard 110 so as to provide a comfortable position for hydration system 100 for patient 502 and to reduce the risk of kinking of hose 104.

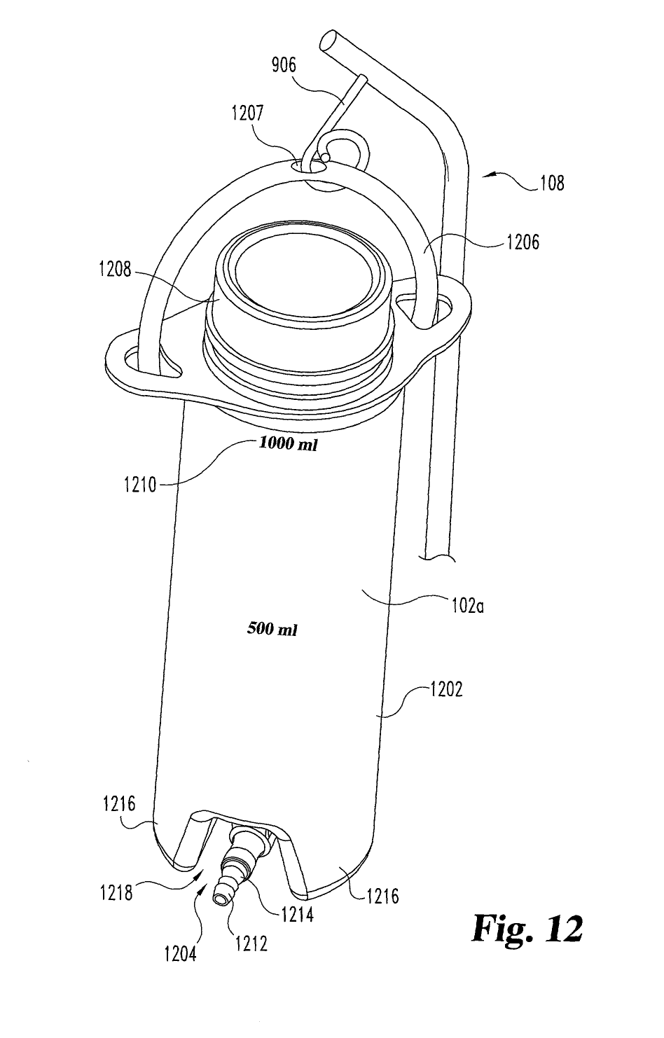

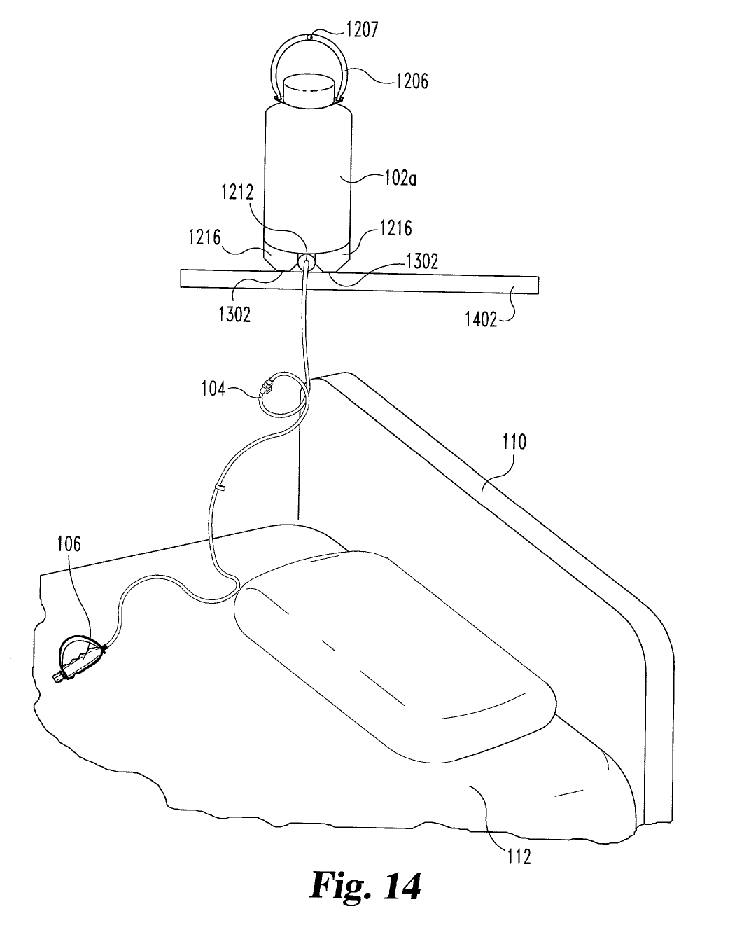

In certain situations, such as during patient transport, a hook may not be available to hang container 102 in order to provide gravity feeding of fluid. Fluid container 102a according to another embodiment of the present invention, which is illustrated in FIGS. 12-15, is adapted to operate in a variety of situations. Fluid container 102a is constructed such that container 102a can be hung (FIG. 12) or stably placed in an upright position on top of a support (FIG. 14), such as a table, while at the same time minimizing the risk of hose 104 kinking. Referring now to FIG. 12, container 102a includes a hollow container body 1202, an outlet end portion 1204, a hanger 1206 with a hanger hole 1207, a cap 1208, and fluid level indicators 1210. Container 102a in the illustrated embodiment has a substantially cylindrical shape and has a hollow interior in which fluid is contained. As illustrated, hanger 1206 has hole 1207 in which hook 906 is attached. Level indicators 1210 indicate the volume of fluid in container 102a. Outlet end portion 1204 includes a nozzle 1212 that radially extends from body 1202. Nozzle 1212 includes a plurality of ridges 1214 that are used to seal the connection between nozzle 1212 and hose 104. Container 102a further includes at outlet end portion 1204 a pair of supports 1216 which are used to support container 102a when placed on a support surface. In one form, supports 1216 are solid such that fluid in container 102a completely drains from nozzle 1212 and does not pool in supports 1216. Nozzle 1212 extends between supports 1216 in outlet cavity 1218 formed between supports 1216.

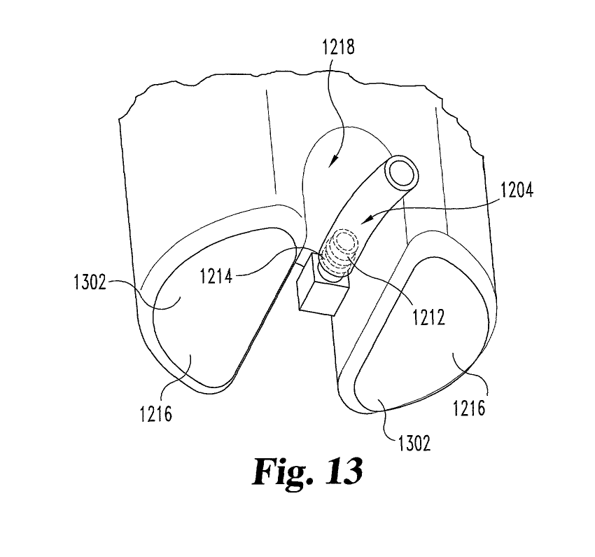

As shown in further detail in FIG. 13, supports 1216 define U-shaped channel 1218 in which nozzle 1212 extends substantially parallel to U-shaped cavity 1218 formed therebetween. Each support 1216 includes a substantially flat support surface 1302 adapted so that container 102a can be stably placed on a surface. By having nozzle 1212 extend in a radial direction from container 102a, the risk of kinking of hose 104 is minimized when container 102a is placed on a support surface. As shown in FIG. 14, container 102a can rest in an upright position on surface 1402 with supports 1216. As illustrated, tube 104 attaches to nozzle 1212 and extends away from container 102a such that hose 104 is not kinked by contact with surface 1402. Container 102a can be either hung or placed on support 1402 above patient 502 such that hydration system 100 is properly pressurized via gravity. In addition, hanger 1206 further allows container 102a to be easily carried during transport of patient 502. The construction of container 102a allows container 102a to be transportable and to be used in a variety of environments.

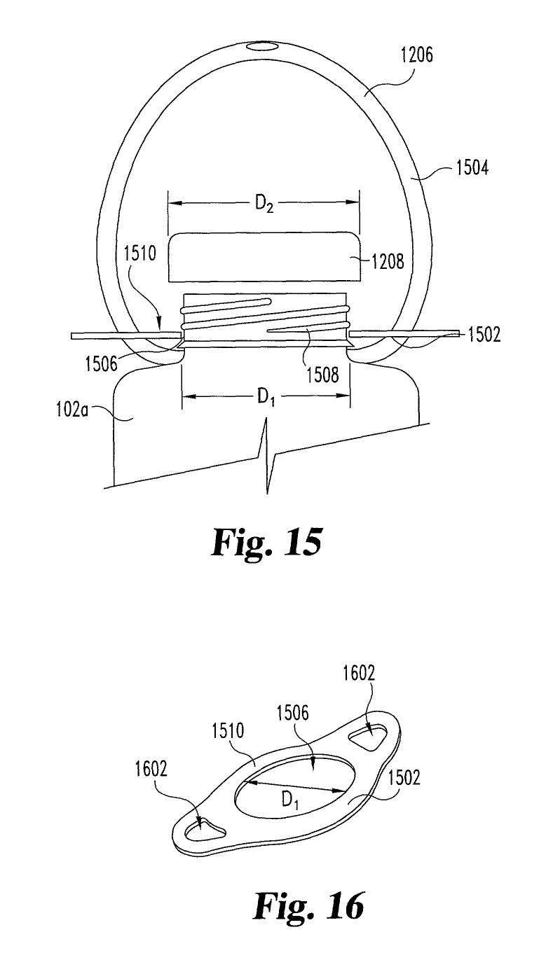

In one embodiment, as shown in FIG. 15, hanger 1206 includes two portions, container coupling portion 1502 and strap portion 1504. Opening 1506 of coupling portion 1502 has a diameter D1 that is large enough to fit around threaded opening 1508 of container 102a, but is smaller than diameter D2 of cap 1208. With this arrangement, cap 1208 secures hanger 1206 to container 102a when threaded onto opening 1508. Seal land portion 1510 of coupling portion 1502 can be used to seal container 102a when cap 1208 is tightly secured against coupling portion 1502. Cap 1208 can be loosened so as to adjust air flow into container 102a, which in turn controls the fluid flow rate from dispenser 106. As illustrated in further detail in FIG. 16, coupling portion 1502 has a pair of strap openings 1602 defined therein in which strap 1504 is secured. Strap openings 1602 allow strap 1504 to be easily replaced if broken.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. An apparatus for hydrating a patient having a hand and a mouth, comprising:
a fluid container;
a fluid supply hose coupled to said fluid container;
a fluid dispenser coupled to said hose, said dispenser being constructed and arranged to control fluid flow from said container and to orally administer fluid to the mouth of the patient; and
a hand strap coupled to said fluid dispenser for securing said fluid dispenser to the hand of the patient to minimize dropping of said fluid dispenser.

2. The apparatus of claim 1, wherein said dispenser includes a mouthpiece adapted to dispense fluid to the patient, said mouthpiece including:
a deformable wall defining at least one fluid opening in said mouthpiece; and
a valve seal provided proximal to said deformable wall to seal said fluid opening when said deformable wall is undeflected from said valve seal and to allow the fluid to flow through said fluid opening when said deformable wall is deflected away from said valve seal.

3. The apparatus of claim 2, wherein said deformable wall is adapted to deflect away from said valve seal when the patient sucks on said mouthpiece.

4. The apparatus of claim 3, wherein:
said mouthpiece includes a bite portion; and
said valve seal includes
a valve seal wall defining a valve seal passageway, and
a flange extending from said valve seal to form a gap between said bite portion and said valve seal wall, said flange being adapted to provide a fulcrum to deflect said deformable wall away from said valve seal when the patient bites on said bite portion.

5. The apparatus of claim 4, wherein said mouthpiece includes a bulbous portion to minimize leakage when the patient drinks from said dispenser.

6. The apparatus of claim 1, wherein:
said dispenser includes
a mouthpiece adapted to dispense the fluid,
a hose connector portion at which said hose is coupled to said dispenser, and
a handle portion positioned between said mouthpiece and said hose connector portion; and
said hand strap spans around said handle portion.

7. The apparatus of claim 6, wherein said dispenser includes a hand strap adjustment portion provided between said mouthpiece and said handle portion to allow said hand strap to laterally move in order to accommodate differently sized hands.

8. The apparatus of claim 6, wherein said hand strap defines a pair of holes at which said hand strap is rotatably couple to said dispenser to permit ambidextrous use of said dispenser.

9. The apparatus of claim 8, wherein said handle portion includes a plurality of indentations adapted to conform to the hand.

10. The apparatus of claim 1, wherein said container includes:
a pair of supports adapted to support said container in an upright position on a support surface;
an outlet nozzle coupled to said hose, said outlet nozzle extending in a radial direction between said pair of supports; and
a hanger member coupled to said container for hanging said container.

11. The apparatus of claim 1, further comprising:
a base defining a bore and a plurality of radially extending slots, said base having a pair of opposing side walls with a clamp adapted to clamp to a support and to provide lateral positional adjustment along said support;
a riser including a rod and a pin extending from said rod, said rod having a portion received in said bore and said pin being received in one of said slots to provide rotational positioning;
a hook coupled to said riser; and
wherein said container is hung from said hook.

12. A fluid dispenser, comprising:
a mouthpiece defining a fluid passage, said mouthpiece including
a deformable wall extending across said fluid passage, said deformable wall defining at least one fluid opening in said mouthpiece adapted to dispense fluid to a patient, and
a valve seal provided within said fluid passage to seal said fluid opening when said deformable wall is undeflected from said valve seal and to allow the fluid to flow through said fluid opening when said deformable wall is deflected away from said valve seal.

13. The apparatus of claim 12, wherein said deformable wall is adapted to deflect away from said valve seal when the patient sucks on said mouthpiece.

14. The apparatus of claim 12, wherein:
said mouthpiece includes a bite portion; and
said valve seal includes
a valve seal wall defining a valve seal passageway, and
a flange extending from said valve seal, said flange being adapted to provide a fulcrum to deflect said deformable wall away from said valve seal when the patient bites on said bite portion.

15. The apparatus of claim 14, wherein said valve seal wall has a cylindrical shape and is positioned within said bite portion to reduce collapsing of said fluid passage during biting.

16. The apparatus of claim 12, wherein said mouthpiece includes a bulbous portion to minimize leakage when the patient drinks from said dispenser.

17. The apparatus of claim 12, further comprising:
a hose connector portion positioned opposite said mouthpiece, said hose connector being adapted to couple to a hose;
a handle portion positioned between said mouthpiece and said hose connector portion; and
a hand strap coupled to said hose connector portion and spanning around said handle portion.

18. A hanger assembly for supporting a fluid container used to hydrate a patient, comprising:
a base including a connector wall and a pair of opposing side walls extending from said connector wall to straddle a support, said base further including a clamping mechanism adapted to secure said base to said support and to allow lateral positional adjustment of said base along said support, said base defining a bore and a plurality of radially extending slots;
a riser including a rod and a pair of opposing pins extending from said rod, said rod having a portion received in said bore and said pins being received in said slots to provide incremental rotational positioning of said riser; and
a hook coupled to said riser to support the fluid container.

19. A fluid container for hydrating a patient, comprising:
a hollow container body adapted to contain fluid, said body including
a pair of supports each having flat support surfaces to stably support said body in an upright position on a support surface, said pair of supports defining an outlet cavity, and
a nozzle extending in a radial direction in said outlet cavity between said pair of supports and being adapted to couple to a hose to minimize kinking of the hose; and
a hanger coupled to said body to hang said body.

20. The fluid container of claim 19, further comprising;
wherein said body has a threaded opening;
a cap threadedly coupled to said threaded opening, said cap having an outer diameter;
wherein said hanger includes a coupling portion and a strap portion, said coupling portion defining a central opening received around said threaded opening and a pair of strap openings in which said strap portion is coupled to said coupling portion, said central opening having an outer diameter less than said outer diameter of said cap, said cap being adjustable relative to said coupling portion to control airflow into said hollow container body.
